(19) United States
(12) Patent Application Publication
(10) Pub. No.: US 2021/0047441 A1
(43) Pub. Date: Feb. 18, 2021

(54) ANTHOCYANIDIN COMPLEX

(71) Applicant: Sapiotec GmbH, Wurzburg (DE)

(72) Inventors: Norbert Roewer, Wurzburg (DE); Jens Broscheit, Wurzburg (DE)

(21) Appl. No.: 16/860,685

(22) Filed: Apr. 28, 2020

Related U.S. Application Data
(63) Continuation of application No. 16/013,243, filed on Jun. 20, 2018, now abandoned.
Application No. 16/013,243 is a continuation of application No. 14/389,474, filed on Sep. 30, 2014, now abandoned.

(30) Foreign Application Priority Data
Mar. 30, 2012 (EP) 12002350.2

(51) Int. Cl. C08B 37/16; C08L 5/16

(52) U.S. Cl. CPC C08B 37/0012; C08L 5/16; C08B 37/0015

(57) ABSTRACT

The invention relates to a complex of pure anthocyanidin and a sulfoalkyl ether β-cyclodextrin, which complex can be formulated as an aqueous solution and as a solid, and a method for producing such a complex. Complexes according to the invention are storage-stable and can be well formulated as an aqueous solution.

### CROSS-REFERENCE TO RELATED APPLICATIONS

This application is a continuation of U.S. patent application Ser. No. 16/013,243, filed Jun. 20, 2018, which a continuation of U.S. patent application Ser. No. 14/389,474, filed Sep. 30, 2014, now abandoned, which is a § 371 U.S. National Entry of International Application No. PCT/EP2013/056707, filed Mar. 28,2013, which claims the benefit of European Patent Application 12002350.2, filed Mar. 30, 2012, each of which is incorporated herein by reference in its entirety.

The invention relates to a complex of an anthocyanidin and a sulfoalkyl ether β-cyclodextrin.

Anthocyanidins are zymochromic pigments which occur in most higher terrestrial plants. Anthocyanidins are sugar-free (aglycones) and closely related to the sugar-containing anthocyanins. Anthocyanidins are pigments and possess antioxidant properties.

The object underlying the invention is to provide anthocyanidins in a form in which they are easy to handle and formulate and are storage-stable.

The object is achieved by a complex of an anthocyanidin and a sulfoalkyl ether β-cyclodextrin.

Some terms used within the context of the invention will first be explained.

Anthocyanidins have the basic structure shown below.

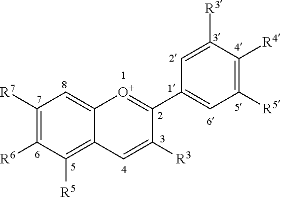

The substituents in this formula are selected from the group consisting of hydrogen, hydroxy group and methoxy group.

Cyclodextrins are cyclic oligosaccharides of glucose molecules linked by an α-1,4-glycosidic bond. β-Cyclodextrin possesses seven glucose units. In the case of a sulfoalkyl ether β-cyclodextrin, hydroxy groups of the glucose unit in a sulfoalkyl alcohol are etherified. According to the invention, generally only some of the 21 hydroxy groups of a β-cyclodextrin are etherified.

The preparation of sulfoalkyl ether cyclodextrins is known to the person skilled in the art and is described, for example, in U.S. Pat. No. 5,134,127 or WO 2009/134347 A2.

Sulfoalkyl ether groups are used in cyclodextrins in the prior art to increase their hydrophilicity or water solubility. The invention has recognized that the sulfoalkyl ether groups contribute to a particular degree to increasing the stability of the complex of anthocyanidins and correspondingly substituted β-cyclodextrin and thus substantially improve the storage stability and formulatability of the anthocyanidins, which are particularly sensitive to oxidation. The complex according to the invention can be formulated as a storage-stable aqueous solution or solid, as will be shown in greater detail below.

Particular preference is given according to the invention to complexing with sulfobutyl ether β-cyclodextrin (SEB-β-CD). A possible explanation for this, which does not limit the scope of protection, is that the negatively charged sulfobutyl units interact electrostatically with the positively charged anthocyanidins and, of the alkyl groups, the butyl group possesses the optimal length for sterically permitting a corresponding interaction.

The degree of substitution of the cyclodextrin with sulfoalkyl ether groups is preferably from 3 to 8, more preferably from 4 to 7. Suitable sulfobutyl ether β-cyclodextrins having a mean degree of substitution of from 6 to 7 are described, for example, in the mentioned WO 2009/134347 A2 and are available commercially under the trade name Captisol®. Corresponding cyclodextrins having a degree of substitution of from 4 to 5, for example 4.2, can likewise be used.

The anthocyanidins complexed according to the invention are preferably selected from the group consisting of aurantinidin, cyanidin, delphinidin, europinidin, luteolinidin, pelargonidin, malvidin, peonidin, petunidin and rosinidin. The chemical structure corresponds to formula I given above with the following substitution pattern

| | R^{3′} | R^{4′} | R^{5′} | R³ | R⁵ | R⁶ | R⁷ |
|---|---|---|---|---|---|---|---|
| Aurantinidin | —H | —OH | —H | —OH | —OH | —OH | —OH |
| Cyanidin | —OH | —OH | —H | —OH | —OH | —H | —OH |
| Delphinidin | —OH | —OH | —OH | —OH | —OH | —H | —OH |
| Europinidin | —OCH₃ | —OH | —OH | —OH | —OCH₃ | —H | —OH |
| Luteolinidin | —OH | —OH | —H | —OH | —OH | —H | —OH |
| Pelargonidin | —H | —OH | —H | —OH | —OH | —H | —OH |
| Malvidin | —OCH₃ | —OH | —OCH₃ | —OH | —OH | —H | —OH |
| Peonidin | —OCH₃ | —OH | —H | —OH | —OH | —H | —OH |
| Petunidin | —OH | —OH | —OCH₃ | —OH | —OH | —H | —OH |
| Rosinidin | —OCH₃ | —OH | —H | —OH | —OH | —H | —OCH₃ |

Particular preference is given within the context of the invention to a complex with delphinidin.

The invention further provides an aqueous solution of a complex according to the invention.

There is further provided a process for the preparation of such a complex and of a corresponding aqueous solution, comprising the steps:
a) preparing an aqueous solution of the sulfoalkyl ether β-cyclodextrin,
b) adding the anthocyanidin and mixing to prepare the complex.

In step a) there is preferably prepared an aqueous solution which comprises from 5 to 10% by weight of the cyclodextrin that is used. It is particularly preferred within the context of the invention if the pH of the aqueous solution is adjusted during or after, but preferably before, the addition of the anthocyanidin, preferably delphinidin, to a pH of 7 or less, preferably 6 or less, more preferably 5 or less, more preferably from 4 to 5. It has been shown that, at this pH, a higher concentration of the complex in aqueous solution can be established.

The concentration of the anthocyanidin, calculated as chloride, is preferably at least 0.5 mg/ml, more preferably at least 1.0 mg/ml, more preferably at least 1.5 mg/ml, more preferably 2.0 mg/ml. Within the context of a preferred embodiment, the particularly preferred concentration range of at least 2.0 mg/ml can be established in particular in a aqueous solution having a pH of from 4 to 5.

Within the context of the preparation according to the invention, mixing of the constituents of the aqueous solution can be carried out by stirring, preferred times for mixing are from 2 to 20 hours. The operation is preferably carried out in the dark in order to avoid light-induced oxidation.

The invention further provides a solid comprising a complex according to the invention, which solid is obtainable according to the invention by removing the solvent from an aqueous solution according to the invention. The removal can preferably be carried out by freeze-drying (lyophilization). Both the aqueous solution according to the invention and the solid possess high storage stability.

Embodiments of the invention are described below.

1. Materials used:

The following cyclodextrins are used:

| | |
|---|---|
| α-CD | ID No: CYL-2322 |
| β-CD | ID No: CYL-3190 |
| γ-CD | ID No: CYL-2323 |
| (2-Hydroxypropyl)-β-CD | ID No: L-043/07 |
| Sulfobutyl ether β-CD | ID No: 47K010111 |

Delphinidin chloride was obtained from Extrasynthese.

30

2. Determination of the delphinidin content

A reverse phase HPLC process was used for determining the content of delphinidin chloride in the delphinidin-containing compositions. The following reagents were used thereby:

Purified water

Methanol for the chromatography

Formic acid, p.a.

1 M hydrochloric acid as volumetric solution.

The column used was a Waters X Bridge™ C18, 35 μ1, 150 mm×4.6 mm.

The mobile phases were as follows:

Channel A: water 950 ml, methanol 50 ml, formic acid 10 ml

Channel B: water 50 ml, methanol 950 ml, formic acid 10 ml

The following gradient program was used:

| Time [min] | Percent channel B |
|---|---|
| 0 | 0 |
| 5 | 0 |
| 25 | 60 |
| 30 | 100 |

Stop time: 35 minutes

Post time: 8 minutes

Flow rate: 1 ml/min

Injection volume: 20 μl

Column temperature: 30° C. +/−2° C.

UV-Vis detector: 530 μm for the assay, 275 μm for the detection of impurities

Integrator: area

Solutions and sample preparation:

Dilution solution 1: mixture of 100 ml of methanol and 2.6 ml of 1 M HCl

Dilution solution 2: mixture of 100 ml of 40 percent methanol and 2.6 ml of 1 M HCl

Calibration solution: A reference solution of delphinidin was prepared by weighing 10 mg of delphinidin chloride into a 10 ml flask and dissolving it in dilution solution 1. After the dissolution, the solution was diluted approximately 10-fold with dilution solution 2 in order to produce an approximate concentration of 0.1 mg/ml.

The control calibration solution was prepared in the same manner. The calibration solutions were analyzed immediately by means of HPLC because delphinidin chloride is unstable in solution.

Preparation of the test solutions:

In order to determine the delphinidin content of solids prepared according to the invention (for preparation see below), approximately 50 mg of the composition were weighed into a 10 ml flask. The composition was then diluted in dilution solution 2 and diluted further with the same dilution solution 2 until an approximate delphinidin concentration of 0.1 mg/ml was established.

The determination of the delphinidin content in the samples was calculated with the aid of Agilent ChemStation software using calibration with the described external standard.

### EXAMPLE 1

Complexing of delphinidin with SBE-β-CD.

In this example, the complexing of delphinidin by various cyclodextrins and the solubility of the complex in aqueous solution are studied. Complexing with SBE-β-CD is in accordance with the invention, the other tests on different cyclodextrins or solubility of delphinidin (uncomplexed) are comparative tests.

Neutral aqueous solutions comprising 10% by weight of the respective cyclodextrin were prepared. In the case of β-CD, a concentration of only 2% by weight was chosen on account of its poor solubility.

In each case 5 ml of the aqueous cyclodextrin solutions and of pure water were introduced into glass flasks. An excess of delphinidin chloride was then added. The required excess amount was 10 mg for the solutions of α-, β- and γ-cyclodextrin and 15 mg for the solutions of HPBCD (2-hydroxypropyl-β-cyclodextrin) and SBE-β-CD.

The suspensions were stirred for 20 hours at 30° C. in the dark. They were then filtered through a membrane filter of 0.22 μm pore size.

The achievable solubilities are shown in Table 1 below.

| | Cyclodextrin | Delphinidin |
|---|---|---|
| Cyclodextrin | concentration | chloride |
| — | 0 | 0.07 mg/ml |
| α-CD | 10% | 0.14 mg/ml |
| β-CD | 2% | 0.05 mg/ml |
| γ-CD | 10% | 0.21 mg/ml |
| HPBCD | 10% | 0.19 mg/ml |
| SBE-β-CD | 10% | 0.66 mg/ml |

It will be seen that the complexing and the increase in solubility effected thereby is far better for SBE-β-CD than for the other cyclodextrins.

### EXAMPLE 2

### Influence of the pH

In this example, the influence of the pH on the solubility of a delphinidin-SBE-β-CD in aqueous solution was studied. Aqueous solutions of SEB-β-CD were prepared according to the procedure of Example 1, but these solutions were adjusted with 1 M HCl to the acid pH values mentioned in Table 2. Delphinidin chloride was then added according to the procedure of Example 1 and further processing was carried out, the only difference being that the stirring time was limited to 2.5 hours. The results are shown in Table 2 below.

| | Delphinidin |
|---|---|
| pH | chloride |
| 6.0 | 0.60 mg/ml |
| 4.8 | 2.12 mg/ml |
| 4.1 | 2.03 mg/ml |

It will be seen that, at pH values of from 4 to 5, the solubility of the complexed delphinidin chloride increases by a factor of approximately 3 compared with the neutral pH.

### EXAMPLE 3

### Preparation of a Solid According to the Invention

In this example, a complex according to the invention is formulated as a solid. For comparison purposes, a delphinidin/HPBCD complex and a delphinidin/starch formulation are prepared in the form of a solid.

### Example 3.1

### Delphinidin/SBE-β-CD

5 g of SEB-β-CD were dissolved in 40 ml of distilled water to give a clear solution. The pH of the solution was adjusted to 4.8 by means of 1 M HCl. 0.11 g of delphinidin chloride was then added, and stirring was carried out for 2 hours at 27° C. in the dark. The homogeneous liquid was vacuum filtered through a cellulose nitrate membrane filter having a pore size of 0.45 μm. The solution was frozen and then freeze-dried at −48° C. and a pressure of approximately 10.3 Pa (77 mTorr). The lyophilizate was ground and sieved through a sieve of 0.3 mm mesh size.

### Example 3.2

### Delphinidin/HPBCD

The procedure was as in Example 3.1, but a significant amount of material was filtered off during the filtration, which indicates that the solubilization was significantly less effective than in the case of the use of SBE-β-CD according to Example 3.1.

### EXAMPLE 3.3

### Delphinidin/Starch Formulation

5 g of starch were suspended in 40 ml of distilled water. A white suspension was obtained. The pH of the solution was adjusted to 4.6 with 1 M HCl. 0.11 g of delphinidin chloride was then added, and stirring was carried out for 2 hours at 27° C. in the dark. The homogeneous liquid obtained was freeze-dried, ground and sieved as in Example 3.1.

Example 3.1 is in accordance with the invention, Examples 3.2 and 3.3 are comparative examples.

### EXAMPLE 4

### Stability Tests

The solids according to Examples 3.1 to 3.3 were stored under the following conditions:

8 days at room temperature in brown glass bottles with a screw fastening,

then 22 days at room temperature in glass containers under an oxygen atmosphere in the dark.

The last 22 days of the above-described storage were carried out in glass vials having a volume of 20 ml. 250 ml of each of the samples previously already stored for 8 days were introduced therein, and the vials were closed with a rubber stopper and sealed. The head space of the vials was flushed with pure oxygen by means of two injection needles. The samples were then stored in the dark.

The delphinidin content of the solids (calculated as delphinidin chloride and indicated in % by weight) was determined by means of the HPLC method described above. The results are to be found in Table 3 below.

| | Time elapsed [days] | | | | |
|---|---|---|---|---|---|
| | Start | 2 | 8 | 19 | 30 |
| Example 3.1 | 1.69 | 1.52 | 1.55 | 1.40 | 0.93 |
| Example 3.2 | 1.30 | 1.20 | 1.14 | 1.03 | 0.68 |
| Example 3.3 | 1.60 | 1.59 | 1.56 | 1.53 | 1.15 |

The results show that it is possible according to the invention to prepare a delphinidin complex which possesses high stability and thus good storage stability even under a pure oxygen atmosphere. The complex further possesses good solubility in aqueous, in particular slightly acidic solutions, so that delphinidin can be formulated in various ways according to the invention. The stability of the solid according to the invention is similarly good to that of a formulation with starch (Example 3.3), but that comparative example cannot be formulated as an aqueous solution.

### EXAMPLE 5

### Stability Tests in Aqueous Solution

In order to determine the content of delphinidin chloride in the delphinidin-containing solutions, a reverse phase HPLC process similar to that already described above was used. The following reagents were used thereby:

Purified water

Methanol for the chromatography

Formic acid, p.a.

1 M hydrochloric acid as volumetric solution.

The column used was a Waters X Bridge™ C18, 35 μ1, 150 mm×4.6 mm.

The mobile phases were as follows:

Channel A: water 770 ml, methanol 230 ml, formic acid 10 ml

Channel B: water 50 ml, methanol 950 ml, formic acid 10 ml

The following gradient program was used:

| Time [min] | Percent channel B |
|---|---|
| 0 | 0 |
| 5 | 0 |
| 20 | 20 |
| 25 | 100 |

Stop time: 25 minutes

Post time: 8 minutes

Flow rate: 1 ml/min

Injection volume: 20 μl

Column temperature: 30° C. +/−2° C.

UV-Vis detector: 530 μm for the assay, 275 μm for the detection of impurities

Integrator: area

Solutions and sample preparation:

Dilution solution 1: mixture of 100 ml of methanol and 2.6 ml of 1 M HCl

Dilution solution 2: mixture of 100 ml of 50% methanol and 2.6 ml of 1 M HCl

Calibration solution: A reference solution of delphinidin was prepared by weighing 10 mg of delphinidin chloride into a 10 ml flask and dissolving it in dilution solution 1. After the dissolution, the solution was diluted approximately 10-fold with dilution solution 2 in order to produce an approximate concentration of 0.1 mg/ml.

The control calibration solution was prepared in the same manner. The calibration solutions were analyzed immediately by means of HPLC because delphinidin chloride is unstable in solution.

Preparation of the test solutions:

In order to determine the delphinidin content of an aqueous solution according to the invention, delphinidin/SBE-β-CD of Example 3.1 (according to the invention) and delphinidin (comparative example were dissolved in 0.9% NaCl solution until a starting concentration (based on the delphinidin) of 1.584 mg/ml (example according to the invention) and 0.0216 mg/ml (comparative example) had been established. The solutions were prepared at room temperature and then stored at 37° C. in the dark in closed vials.

The delphinidin content was determined after 1, 2, 3 and 4 hours. The table below shows the calculated content as the percentage of the above-mentioned starting concentration.

| | Delphinidin | Delphinidin/ |
|---|---|---|
| Time [h] | uncomplexed | SBE-β-CD |
| 0 | 100% | 100% |
| 1 | 8.3% | 80.7% |
| 2 | 6.5% | 74.5% |
| 3 | 5.6% | 64.7% |
| 4 | 5.1% | 62.8% |

The determination of the delphinidin content in the samples was calculated with the aid of Agilent ChemStation software using calibration with the described external standard.

1. A complex of an anthocyanidin and a sulfoalkyl ether β-cyclodextrin.

2. The complex as claimed in claim 1, wherein the sulfoalkyl ether β-cyclodextrin is a sulfobutyl ether β-cyclodextrin (SBE-β-CD).

3. The complex as claimed in claim 1, wherein the degree of substitution of the cyclodextrin with sulfoalkyl ether groups is from 3 to 8.

4. The complex as claimed in claim 1, wherein the anthocyanidin is selected from the group consisting of aurantinidin, cyanidin, delphinidin, europinidin, luteolinidin, pelargonidin, malvidin, peonidin, petunidin and rosinidin.

5. The complex as claimed in claim 4, wherein the anthocyanidin is delphinidin.

6. An aqueous solution of a complex as claimed in claim 1.

7. The aqueous solution as claimed in claim 6, wherein said aqueous solution has a pH of 7 or less.

8. The aqueous solution as claimed in claim 6, wherein the anthocyanidin is at a concentration, calculated as chloride, of at least 0.5 mg/ml.

9. A solid comprising a complex of an anthocyanidin and a sulfoalkyl ether β-cyclodextrin, obtainable by removing solvent from an aqueous solution as claimed in claim 6.

10. A process for the preparation of a complex of an anthocyanidin and a sulfoalkyl ether β-cyclodextrin, comprising the steps:
a) preparing an aqueous solution of the sulfoalkyl ether β-cyclodextrin,
b) adding the anthocyanidin and mixing to prepare the complex.

11. The process as claimed in claim 10, wherein the solution prepared in step a) comprises from 5 to 10% by weight of the sulfoalkyl ether β-cyclodextrin.

12. The process as claimed in claim 10, wherein the pH of the solution prepared in step a) is adjusted before the addition of the anthocyanidin to a pH of 7 or less.

13. The process as claimed in claim 10, wherein the mixing in step b) takes place over a period of from 2 to 20 hours.

14. The complex as claimed in claim 3, wherein the degree of substitution of the cyclodextrin with sulfoalkyl ether groups is from 4 to 7.

15. The aqueous solution as claimed in claim 6, wherein said aqueous solution has a pH of 6 or less.

16. The aqueous solution as claimed in claim 6, wherein said aqueous solution has a pH of 5 or less.

17. The aqueous solution as claimed in claim 6, wherein said aqueous solution has a pH from 4 to 5.

18. The aqueous solution as claimed in claim 6, wherein the anthocyanidin is at a concentration, calculated as chloride, of at least 1.0 mg/ml.

19. The aqueous solution as claimed in claim 6, wherein the anthocyanidin is at a concentration, calculated as chloride, of at least 1.5 mg/ml.

20. The aqueous solution as claimed in claim 6, wherein the anthocyanidin is at a concentration, calculated as chloride, of at least 2.0 mg/ml.
